(19) United States
(12) Patent Application Publication
(10) Pub. No.: US 2025/0018081 A1
(43) Pub. Date: Jan. 16, 2025

(54) ELECTRIC FIELD STERILIZER FOR PATHOGENS

(71) Applicant: HYPERTEC SYSTÈMES INC., Saint-Laurent (CA)

(72) Inventors: Mathieu Ludovic MALBOEUF JOSET, Coteau-du-Lac (CA); Andrew PIASKOWSKI, Saint-Colomban (CA); Eliot AHDOOT, Dollard-des-Ormeaux (CA); Robert AHDOOT, Dollard-des-Ormeaux (CA)

(21) Appl. No.: 18/701,203

(22) PCT Filed: Oct. 17, 2022
(86) PCT No.: PCT/CA22/51526

§ 371 Date: Apr. 12, 2024

Related U.S. Application Data
(60) Provisional application No. 63/256,358, filed on Oct. 15, 2021.

(51) Int. Cl. A61L 9/16; A61L 2/18; A61L 2/20; A61L 2/26; A61L 9/015; A61L 9/14

(52) U.S. Cl. CPC A61L 9/16; A61L 2/186; A61L 2/202; A61L 2/26; A61L 9/015; A61L 9/145; A61L 2202/11; A61L 2209/211; A61L 2209/212

(57) ABSTRACT

The sterilization device for pathogens includes a housing having a first end and a second end opposite to the first end, the first end having formed therein at least one air inlet and the second end having formed therein at least one air outlet, a capacitive component disposed within the housing and extending along an air flow path disposed between the at least one air inlet and air outlet, a voltage source electrically connected to the capacitive component, the voltage source configured to supply an alternating voltage to the capacitive component for causing the capacitive component to generate an electric field, and a positive displacement device disposed within the housing and configured to direct a flow of air through the at least one air inlet and towards the capacitive component. One or more pathogens present in the flow of air are destroyed by the electric field.

### CROSS-REFERENCE TO RELATED APPLICATIONS

The present patent application is a United States National Stage of International Application No. PCT/CA2022/051526, filed on Oct. 17, 2022, which claims priority on U.S. Patent Application No. 63/256,358, filed on Oct. 15, 2021, the entire contents of which are incorporated herein by reference.

### TECHNICAL FIELD

The present disclosure relates generally to the field of sterilization of pathogens.

### BACKGROUND

Several technologies have been used for the purposes of destroying pathogens, such as viruses and bacteria. Existing devices may for example employ microwave radiation, or radio frequency (RF) molecular or particulate resonance. However, these technologies generally fail to achieve a high kill efficacy and may be less effective at sterilizing airborne pathogens, which are mainly carried by solid or liquid particulates such as dust and, more commonly, liquid or water aerosols suspended in the air. Other known devices use radiation technologies, such as ultraviolet-C(UVC) and plasma radiation. While UVC radiation has been found to be relatively effective at destroying surface pathogens, this type of radiation is less effective at destroying airborne pathogens. Indeed, extended periods of exposure are generally required for a pathogen to acquire a dose of radiation sufficient to destroy the pathogen. However, pathogens typically move in a rapid air flow through a chamber of existing UVC sterilization devices and are exposed to UVC radiation for a short period of time (i.e. fractions of a second). In order to improve efficacy, multiple passes of the same pathogen through the device's chamber would be necessary for the pathogen to receive a sufficient dose of radiation to achieve its denaturisation.

### SUMMARY

In accordance with one aspect, there is provided a sterilization device for pathogens, comprising: a housing having a first end and a second end opposite to the first end, the first end having formed therein at least one air inlet and the second end having formed therein at least one air outlet; a capacitive component disposed within the housing and extending at least partially along an air flow path disposed between the at least one air inlet and the at least one air outlet; a voltage source electrically connected to the capacitive component, the voltage source configured to supply an alternating voltage to the capacitive component for causing the capacitive component to generate an electric field, and a positive displacement device disposed within the housing and configured to direct a flow of air through the at least one air inlet and towards the capacitive component, wherein one or more pathogens present in the flow of air are destroyed by the electric field.

The sterilization device and method as described herein may further include one or more of the following features, in whole or in part, and in any combination.

In some embodiments, the voltage source is configured to supply the alternating voltage to the capacitive component for causing the capacitive component to generate the electric field by electrical resonance.

In some embodiments, the one or more pathogens are destroyed by current induced through the one or more pathogens by the electric field.

In some embodiments, the air outlet is configured to discharge therethrough a sterilized volume of air produced at an exit of the capacitive component, the sterilized volume of air devoid of any live pathogens.

In some embodiments, the capacitive component comprises a first capacitor element and at least one second capacitor element, the first capacitor element and the at least one second capacitor element being cylindrical-shaped and electrically conductive, the first capacitor element and the at least one second capacitor element arranged concentrically along the air flow path from the at least one air inlet to the at least one air outlet.

In some embodiments, the device further comprises a separating member interposed between the first capacitor element and the at least one second capacitor element for spacing the first capacitor element from the at least one second capacitor element.

In some embodiments, the separating member is an insulator.

In some embodiments, the insulator is resistant to a corona discharge caused by ionization of air within the housing.

In some embodiments, the separating member comprises a quartz tube.

In some embodiments, the first capacitor element is arranged concentrically within the at least one second capacitor element, and a blocking member is provided at an end of the first capacitor element, the end exposed to the flow of air directed by the positive displacement device towards the capacitive component.

In some embodiments, the blocking member is configured to prevent the flow of air from being directed into the first capacitor element via the end of the first capacitor element, and to allow the flow of air to be directed towards a space defined between an outer wall of the first capacitor element and an inner wall of the at least one second capacitor element.

In some embodiments, the electric field is generated in the space defined between an outer wall of the first capacitor element and an inner wall of the at least one second capacitor element, and the blocking member is configured to prevent the flow of air from being directed into a space defined by inner walls of the first capacitor element and where no electric field is present to destroy the pathogens.

In some embodiments, the at least one second capacitor element comprises a single second capacitor element, and a first terminal of the voltage source is connected to the first capacitor element and a second terminal of the voltage source is connected to the second capacitor element.

In some embodiments, the at least one second capacitor element comprises a plurality of second capacitor elements, and terminals of the voltage source are connected to the first capacitor element and to the plurality of second capacitor elements in alternation.

In some embodiments, the electric field generated by the capacitive component and liquid droplets and/or solid particles present in surrounding air cause at least one chemical compound to be produced within the housing, the at least one chemical compound adapted to destroy the one or more pathogens present in the flow of air and one or more pathogens present on at least one surface external to the device.

In some embodiments, the at least one chemical compound comprises ozone and/or hydrogen peroxide.

In some embodiments, the at least one chemical compound is adapted to be discharged through the at least one air outlet and to react with the one or more pathogens present on the at least one surface external to the device.

There is also provided a sterilization device comprising a housing having a first end and a second end opposite to the first end, the first end having formed therein at least one air inlet and the second end having formed therein at least one air outlet, a fan disposed within the housing adjacent the at least one air inlet, a capacitive component disposed within the housing adjacent the fan, along an air flow path from the at least one air inlet to the at least one air outlet, and a voltage source electrically connected to the capacitive component, the voltage source configured to supply an alternating voltage to the capacitive component for causing the capacitive component to generate an electric field. The fan is configured to direct a flow of air received through the at least one air inlet towards the capacitive component to cause one or more pathogens present in the flow of air to be destroyed by the electric field.

In accordance with another aspect, there is provided a method of sterilizing pathogens, comprising: directing a flow of air through an air passage from an air inlet to an air outlet, and exposing the flow of air to an electric field generated within the air passage, between the air inlet and the air outlet, by supplying an alternating voltage to a capacitive component, the capacitive component producing the electric field. One or more pathogens present in the flow of air are destroyed by the electric field when exposed thereto as the flow of air passes through the air passage.

There is also provided a method comprising providing a housing having a first end and a second end opposite to the first end, the first end having formed therein at least one air inlet and the second end having formed therein at least one air outlet, disposing a fan within the housing adjacent the at least one air inlet, disposing a capacitive component within the housing adjacent the fan, along an air flow path from the at least one air inlet to the at least one air outlet, and electrically connecting a voltage source to the capacitive component, the voltage source configured to supply an alternating voltage to the capacitive component for causing the capacitive component to generate an electric field, the fan configured to direct a flow of air received through the at least one air inlet towards the capacitive component to cause one or more pathogens present in the flow of air to be destroyed by the electric field.

There is also provided a method comprising providing a housing having a first end and a second end opposite to the first end, the first end having formed therein at least one air inlet and the second end having formed therein at least one air outlet, disposing a fan within the housing adjacent the at least one air inlet, disposing a capacitive component within the housing adjacent the fan, along an air flow path from the at least one air inlet to the at least one air outlet, and electrically connecting a voltage source to the capacitive component, the voltage source configured to supply an alternating voltage to the capacitive component for causing the capacitive component to generate an electric field, the fan configured to direct a flow of air received through the at least one air inlet towards the capacitive component to cause one or more airborne pathogens present in the flow of air and/or one or more surface pathogens present on at least one surface external to the housing to be chemically destroyed by chemical compounds created by the electric field and liquid droplets and/or solid particles present in surrounding air.

There is also provided a method comprising directing a flow of air through at least one air passage defined within a housing, the housing having a first end defining at least one air inlet and a second end opposite to the first end defining at least one air outlet, and exposing the flow of air to an electric field generated within the at least one air passage, between the at least one air inlet and the at least one air outlet, by supplying an alternating voltage to a capacitive component disposed within the housing, the capacitive component generating the electric field. As the flow of air passes through the housing, one or more airborne pathogens present in the flow of air and/or one or more surface pathogens present on at least one surface of the housing are chemically destroyed by at least one chemical compound produced by the electric field and liquid droplets and/or solid particles present in surrounding air.

Many further features and combinations thereof concerning embodiments described herein will appear to those skilled in the art following a reading of the instant disclosure.

### BRIEF DESCRIPTION OF THE FIGURES

Reference is now made to the accompanying figures, in which:

FIG. 1A is a schematic diagram of an example sterilization device 100 for treating pathogens, in accordance with one embodiment;

FIG. 1B is a photograph of the example sterilization device 100 of FIG. 1A, in accordance with one embodiment;

FIG. 2 is a schematic diagram of an example of multiple capacitor elements for a sterilization device, in accordance with another embodiment; and

FIG. 3A is a circuit diagram of an example circuit for modelling a sterilization device, in accordance with embodiments of FIG. 1 and FIG. 2; and

FIG. 3B is a photograph of an example implementation of the circuit of FIG. 3A.

### DETAILED DESCRIPTION

Described herein are systems and methods for treating pathogens using electric fields, for example high intensity, alternating electric fields, to cause several effects including dielectric heating. As will be discussed further below, it is proposed herein to denature airborne and/or surface pathogens using high intensity alternating electric fields.

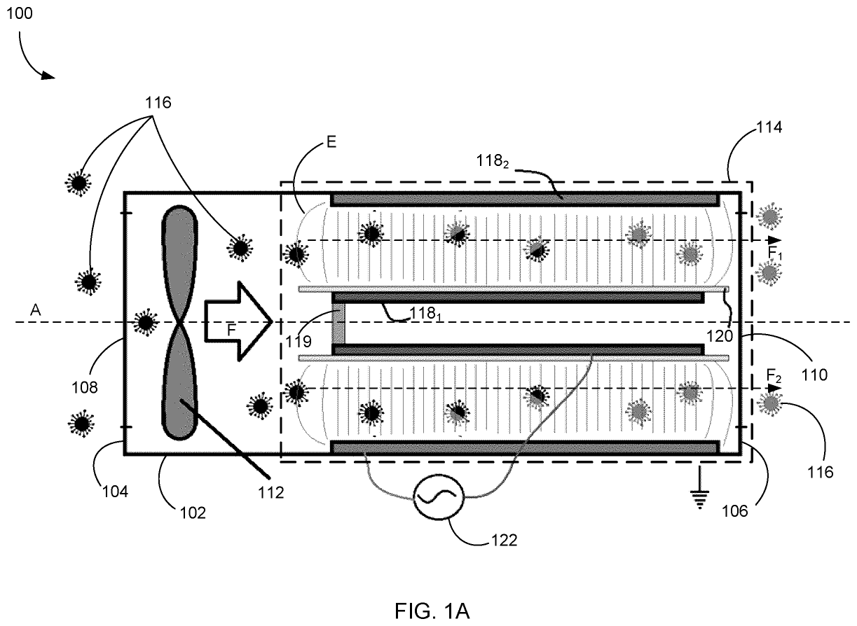

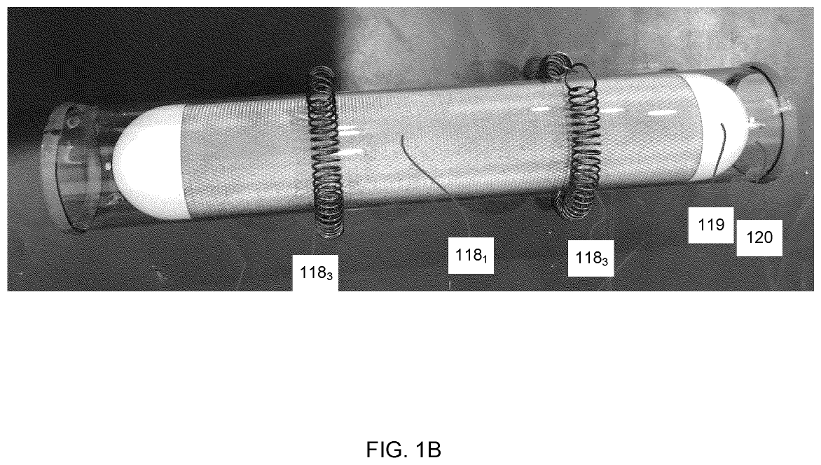

FIG. 1A shows an example of a sterilization device 100 (also referred to herein simply as the “device” 100) for treating airborne and/or surface pathogens. As used herein, the term “airborne pathogens” refers to pathogens (e.g., viruses and bacteria) present in a given volume of air. The term “surface pathogens” refers to pathogens present on one or more surfaces (e.g., an object's surface). The pathogens have similar properties to body tissue and each has a percentage of given content (e.g., 50%˜80% of water). The given liquid within a given pathogen can be considered as an electrolyte having a resistivity in the range of between about 5Ω·m and 1000Ω·m. As known to those skilled in the art, the size range of pathogens varies from less than 10 nm to several μm, with the smallest of pathogens (e.g. parvovirus) having a size in the order of about 20 nm and the largest of pathogens (e.g., bacterium) having a size in the order of about 2 μm. The smallest water droplet size capable of transporting pathogens can be in the order of 100 nm.

In the illustrated embodiment, the device 100 comprises an elongated housing 102 having a first end 104 and a second end 106 opposite the first end 104. At least one air inlet 108 is provided at the first end 104 and at least one air outlet 110 is provided at the second end 106, with a flow path of air being formed from the at least one air inlet 108 to the at least one air outlet 110. The air flow path is along a direction substantially parallel with a central axis A of the device 100. Any suitable means may be used to create openings in the housing 102 for providing the at least one air inlet 108 and the at least one air outlet 110. In addition, the at least one air inlet 108 and the at least one air outlet 110 may have any suitable shape that allows air to flow into and out of the device 100. The device 100 further comprises a positive displacement device 112 (hereinafter referred to as a “fan” 112 for simplicity) for moving air through the housing 102 and an elongated capacitive component (also referred to as a “resonant chamber” or “reactor”) 114. The positive displacement device 112 may be any suitable positive displacement device capable of moving air, including but not limited to, a fan, a compressor, a blower, a diaphragm or the like. Several such positive displacement devices may also be used if required. The fan 112 and the capacitive component 114 are sequentially disposed within the housing 102, along the air flow path.

As will be discussed further below, the capacitive component 114 is configured to generate a high intensity alternating electric field (illustrated by lines E in FIG. 1A) between a first terminal 118₁ of the capacitive component 114, also referred to herein as a “live” or “high voltage” terminal, and a second terminal 118₂ of the capacitive component 114, also referred to herein as a “grounded” terminal. At least one ionizing coil (reference 118₃ in FIG. 1B) may be used to increase the intensity of the alternating electric field (at the surface of a separating member 120 interposed between the capacitors elements 118₁, 118₂) to aid the potential production of additional chemical compounds, including, but not limited to, ozone and hydrogen peroxide. Pathogens as in 116 that are directed into the housing 102 are exposed to the high intensity alternating electric field to cause sterilization of the pathogens 116. As used herein, the term “sterilization” refers to harming one or more target organisms (e.g., pathogens), such that a sterilized volume of air includes a volume of air that is devoid of any live pathogens. The harmed pathogens are referred to herein as denatured or eradicated pathogens.

In one embodiment, the fan 112 is preferably an axial-type fan with an axis of rotation (not shown) substantially parallel to the central axis A. However, any type of positive displacement device suitable for moving air can be used. The fan 112 is disposed adjacent the at least one air inlet 108 and is substantially aligned therewith in order to direct a flow of air introduced into the housing 102 to an internal space of the housing 102. In some embodiments, the device 100 may further comprise an air duct (not shown) positioned adjacent the at least one air inlet 108 and extending therefrom for controlling air flow within the housing 102. The fan 112 is configured to take and generate air flow in a direction substantially parallel to the fan's axis of rotation, at a flow rate and flow velocity required or desirable for the volume of air to be treated, depending on the application. For example, a small room or vehicle may only require lower flow rates to be treated whereas a building would require much larger quantities of air to be treated. As an example only, a hospital room may require 5 air changes per hour (ACH), a bus may require 20 ACH, whereas an airport or office building would require significantly more airflow to be treated. The rate of air flow is such as to ensure that the pathogen as in 116 residing in the air flow has sufficient time to be denatured. In one embodiment, for electric fields having an intensity below 10 KV/cm at a frequency of 60 KHz, it may be desirable for the air flow to be sufficiently slow to effectively denature the pathogen as in 116 (e.g. in about 100 ms). For electric fields having an intensity above 20 KV/cm at a frequency of 60 KHz, it may be desirable for the air flow to be faster, and denaturization may then occur in several milliseconds. It should be understood that each species of pathogen as in 116 will require a different minimum field intensity and frequency to denaturize the pathogen as in 116. Thus, for a broad spectrum of specified pathogens 116, a minimum field intensity at specific minimum frequencies may allow for eradication of all pathogens 116 that are 240 considered.

The flow of air, which contains airborne pathogens as in 116, is directed by the fan 112 towards the capacitive component 114, along a direction (illustrated by arrow F) which is substantially parallel to the central axis A. The air flow remains within the housing 102 for a target amount of time sufficient to cause the pathogens 116 to be destroyed by the high intensity alternating electric field E generated within the capacitive component 114. The pathogens 116 can be thermally destroyed due to the temperature of the water droplets carrying the pathogens 116 being raised (as a result of the high intensity alternating electric field) to a temperature exceeding the temperature at which the pathogens 116 can survive. Using the device 100 may further allow to target the pathogens 116 themselves, since the body of each pathogen 116 is made of a percentage of given content (e.g., 50%˜80% of water). The pathogens 116 may also be ruptured by polarization caused by the high intensity alternating electric field. This results in sterilization of the volume of air in a single pass within the housing 102, the target amount of time may vary depending on the application.

The sterilized volume of air exiting the device 100 via the at least one air outlet 110 (i.e. produced at an exit of the capacitive component 114) is therefore devoid of any live pathogens. The pathogens 116 illustrated in FIG. 1A as being discharged at the at least one air outlet 110 are shown for illustrative purposes only and should be understood as being eradicated pathogens, as shown by their color change, from black (illustrating active pathogens) to grey (illustrating eradicated pathogens).

As will be discussed further below, pathogens as in 116 may also be chemically destroyed by one or more secondary chemical compounds, including, but not limited to, ozone (O₃) and hydrogen peroxide (H₂O₂), created by the high intensity alternating electric field generated in the device 100 and by the liquid droplets and/or solid particles potentially present in the surrounding air. Such liquid droplets may include, for example, humidity present in the ambient air or other types of liquids. In particular, the pathogens 116 may be denaturized by damage caused by the high intensity alternating electric field in the vicinity of ionizing coil 118₃, as well as by the potential generation of additional chemical compounds, including, but not limited to, ozone and hydrogen peroxide by products created by the high intensity alternating electric field. In one embodiment, it is desirable for the intensity of the alternating electric field to be below the ionization level of oxygen and nitrogen molecules.

An electrical discharge (referred to as a “corona discharge” or “corona”) is caused by ionization of air within the device 100, due to the high voltage carried by the capacitive component 114. Depending on the application, the device 100 may therefore be used for destroying surface pathogens in addition (or as an alternative) to destroying airborne pathogens. The device 100 may be operated in two modes, namely a first mode, referred to herein as an “occupancy” mode, and a second mode, referred to herein as a “non-occupancy” mode. In the occupancy mode, the level of potential additional chemical compounds generated by the device 100 may be limited automatically using a local controller (not shown). In the non-occupancy mode, the level of additional chemical compounds may be maximized (e.g., using the controller) to enable rapid sterilization of a closed area. In the non-occupancy mode, sterilization of surfaces in the closed area may indeed be achieved using elevated levels of additional chemical compounds (e.g., levels above about 0.1 ppm). In the occupancy mode, sterilization of surfaces may also be achieved via other chemical compounds created as by-products of the device 100.

Still referring to FIG. 1A, the capacitive component 114 is disposed within the housing 102 adjacent to the fan 112 and extends along a direction substantially parallel to the central axis A of the housing 102. In the illustrated embodiment, the capacitive component 114 comprises a first cylindrical-shaped and electrically conducting capacitor element 118₁ and a second cylindrical-shaped and electrically conducting capacitor element 118₂. The capacitor elements 118₁, 118₂ are concentric and share the same axis (not shown), which is substantially parallel to the central axis A. More specifically, the first capacitor element (also referred to herein as an “inner” capacitor element) 118₁ is positioned concentrically within the second capacitor element (also referred to herein as an “outer” capacitor element) 118₂. In the illustrated embodiment, a blocking member 119 (also referred to as a dielectric barrier) is provided at an end (not shown) of the inner capacitor element 118₁ that is exposed to the flow of air directed by the fan 112. The blocking member 119 may be configured by any suitable means to prevent the flow of air from being directed into the inner capacitor element 118₁. In other words, the blocking member 119 is configured to prevent the flow of air from being directed into a space defined by inner walls of the inner capacitor element 118₁ where no high intensity alternating electric field would be present to destroy the pathogens 116. In this manner, the flow of air is directed into the space provided between the inner capacitor element 118₁ and the outer capacitor element 118₂ (i.e. the space defined by the outer wall of the inner capacitor element 118₁ and the inner wall of the outer capacitor element 118₂), along directions (illustrated by arrows F1 and F2) that are substantially parallel to the central axis A.

In one embodiment, the capacitor elements 118₁, 118₂ are spaced by a separating member 120, which may be any suitable member configured for spacing the capacitor elements 118₁, 118₂ relative to one another to control the discharge of pathogens 116 within the housing 102. The separating member 120 is also an insulator configured to insulate each capacitor element 118₁, 118₂ to avoid arc discharge. In one embodiment, the separating member is made of a material that does not degrade under corona conditions (i.e. is corona-resistant). In one embodiment, the separating member 120 is a quartz tube interposed between the capacitors elements 118₁, 118₂. In some embodiments, the capacitor elements 118₁, 118₂ may alternatively or additionally be spaced by air. Other embodiments may apply. The distance of separation between the capacitor elements 118₁, 118₂ may vary depending on the application, the distance being selected to achieve the desired level of electric field intensity (i.e. the desired field strength) within the device 100.

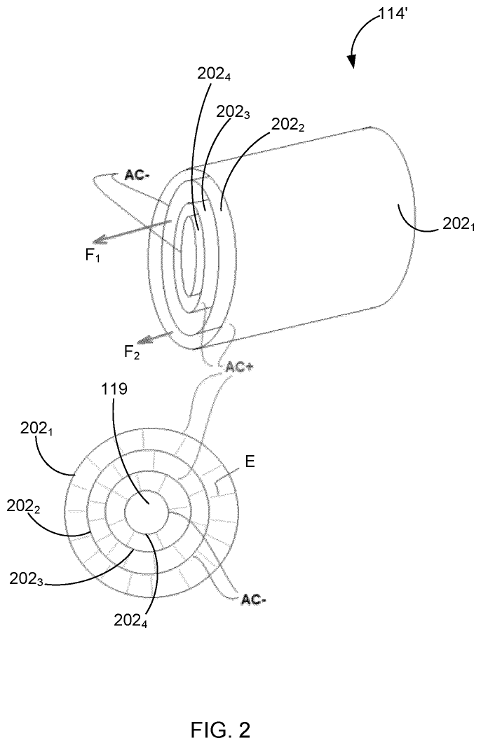

Although FIG. 1A illustrates the capacitive component 114 as comprising two capacitor elements 118₁, 118₂ forming a single capacitor (with the inner capacitor element 118₁ representing one electrode and the outer capacitor element 118₂ representing another electrode), it should be understood that any suitable number of capacitors may apply and that the capacitive component 114 may comprise one or more capacitors. For example, FIG. 2 illustrates an embodiment of a capacitive component 114′ comprising four concentric capacitor elements 202₁, 202₂, 202₃, and 202₄ forming three distinct capacitors. Any suitable multi-capacitor configuration (e.g., comprising a number (N) of concentric capacitor elements together forming N-1 capacitors) may apply, provided each capacitor element has a different potential than an adjacent capacitor element so as to create a high intensity alternating electric field. In addition, while the capacitor elements 118₁, 118₂ and 202₁, 202₂, 202₃, 202₄ are illustrated and described herein as being concentric cylinders, it should be understood that the capacitor elements 118₁, 118₂ may have any other suitable shape (e.g., square, oval, triangle, rectangle, polygon, non-geometrical, and the like) configured to cause generation of an electric field having the desired intensity. For example, the capacitive component 114 or 114′ may include, but is not limited to, plate-shaped capacitor elements. Any suitable configuration of series or parallel circuit topology or air flow direction may be used. Other embodiments may therefore apply.

The device 100 further comprises a voltage source 122 electrically connected to the capacitive component 114 and configured to supply an alternating voltage thereto for the high intensity alternating electric field E to be generated between the walls of the capacitor elements 118₁, 118₂ (i.e. between the outer wall of the inner capacitor element 118₁ and the inner wall of the outer capacitor element 118₂, as illustrated in FIG. 1A). For this purpose, the terminals of the voltage source 122 are electrically connected to each of the capacitor elements 118₁, 118₂, with a first terminal of the voltage source 122 being connected to the outer capacitor element 118₂ and a second terminal of the voltage source 122 being connected to the inner capacitor element 118₁. When the capacitive component 114 comprises more than two capacitor elements, such as in the embodiment illustrated in FIG. 2, the terminals of the voltage source 122 are electrically connected to the capacitor elements in alternation, so as to generate the high intensity alternating electric field E between walls of successive capacitor elements, as shown in FIG. 2. For instance, as shown in FIG. 2, the high voltage (or “live”) terminal (indicated as AC+ in FIG. 2) of the voltage source 122 is connected to the first (or outer) capacitor element 202₁ and to the third capacitor element 202₃, while the grounded terminal (i.e. the terminal referenced to ground potential, indicated as AC—in FIG. 2) of the voltage source 122 is connected to the second capacitor element 202₂ and to the fourth (or inner) capacitor element 202₄.

As used herein, the term “high intensity” refers to an electric field intensity within a range of about 1 KV/cm and greater. In one embodiment, a high intensity electric field has an intensity greater than 10 KV/cm. In another embodiment, the high intensity electric field has an intensity within a range of about 1 KV/cm to about 10 KV/cm. In another embodiment, the high intensity electric field has an intensity within a range of about 10 KV/cm to about 30 KV/cm. Other embodiments may apply. In one particular example, the electric field E generated by the device 100 has an intensity of about 20 KV/cm. The term “high frequency” as used herein with reference to the 370 electric field, which may thus be a high frequency and high intensity electric field, may include any frequency between about 15 KHz to several MHz. In one embodiment, the sterilizing device proposed herein operates at about 60 KHz.

In one embodiment, the proposed device 100 is further configured to provide a power density per unit volume of about 6.5 W/cm³ (or 42.46 W/cm²). In comparison, a typical UVC sterilization device provides a UVC flux having an average power density per unit volume of 0.025 W/cm³ (or 0.088 W/cm²). Therefore, the device 100 may allow to deliver a power density about 482 times greater (by area) and about 260 times greater (by volume) than that delivered by existing UVC sterilization devices. Using the device 100, pathogens as in 116 are therefore exposed to higher energy intensity and power density than in existing UVC sterilization devices. In particular, at a higher frequency and lower field intensity, use of the device 100 can result in the same kill efficacy as a device operating at a lower frequency and higher field intensity.

Generation of the high intensity alternating electric field causes currents (not shown) to be induced through liquids such as water droplets, particulates, and pathogens as in 116 present within the housing 102 of the device 100. These currents in turn cause several effects, dielectric heating as well as biological damage to the exterior protein as well as deoxyribonucleic acid (DNA) and/or ribonucleic acid (RNA) of the pathogens 116, resulting in denaturisation or eradication of the pathogens 116. As used herein, the term “dielectric heating” refers to the process in which a high intensity, high frequency alternating electric field heats a dielectric material. It is proposed herein to use dielectric heating on a particulate without thermal heating of the air mass. For instance, a dielectric loss factor (also referred to as a “dissipation factor”) of Tan (δ) on fine particulates or liquids such as water droplets may be exhibited, the loss factor being indicative of the electrical energy absorbed and lost when the electric field is applied to the particulates or liquids such as water droplets. It is also proposed to exploit the fact that electric fields concentrate in materials of high dielectric constant. Particles suspended in air have higher dielectric constant than the surrounding air. For example, water droplets have a dielectric constant of about 70, compared to air which has a dielectric constant close to unity. In addition, water droplets are polarized and, as the electric field E generated by the capacitive component 114 alternates, polarization of the water droplets present in the housing 102 therefore alternates. This causes a concentrated internal electric current within the housing 102 that heats and/or damages the pathogens 116 carried by the water droplets. Water droplets or biological matter (containing water) also have low resistivity and thus enable large dissipation factors in alternating electric fields, which aids dielectric heating.

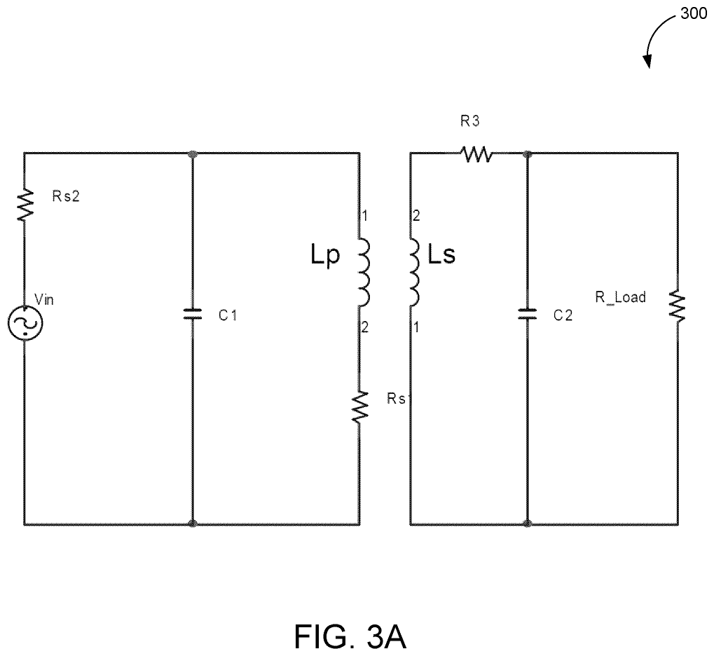

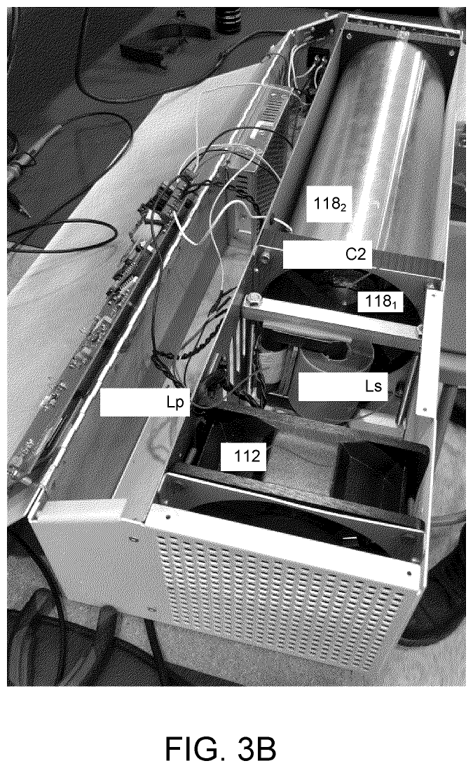

Referring now to FIG. 3A in addition to FIG. 1A, the desired level of electric field intensity may be generated by the device 100 at low power by electrical resonance. As used herein, the term “electrical resonance” refers to the phenomenon whereby, at a particular resonant frequency in an electrical circuit, impedances (or admittances) of elements of the circuit cancel one another. In one embodiment, the power consumed only corresponds to losses in the device 100. In other words, losses in the capacitive component 114 correspond to the power transferred to the particulates or liquids such as water droplets (not shown) carrying the pathogens 116 as well as circuit losses. In one embodiment, this is achieved using a resonant capacitor approach, modeled by the resonant circuit 300 of FIG. 3A, in which the capacitive component 114 is resonant with the secondary inductance of a transformer. In one embodiment, the resonant circuit 300 models a multi-Kilo-Volt Amperes (KVA) resonant transformer/capacitor system with a loose coupling factor (K), the coupling factor being representative of the coupling between the transformer's primary and secondary inductances. The resonant circuit 300 may be used to limit power use and increase efficiency of the device 100. However, in some embodiments, a non-resonant circuit may also apply.

The resonant circuit 300 illustratively comprises a voltage source (having an input voltage Vin) electrically connected in series with an input impedance Rs2, a first capacitor (having a parasitic capacitance C1) electrically connected in parallel with the voltage source, a transformer having a coupling factor K and comprising a primary inductance Lp (having a primary resistance Rs connected in series therewith) and a secondary inductance Ls (having a secondary resistance R3 connected in series therewith), a second capacitor (having a capacitance C2) connected in parallel with the transformer and with an output load R_load. The voltage source models the voltage source 122, the first capacitor is parasitic and does not affect the device's functionality, and the second capacitor C2 models the capacitor between cylinders 118₁ and 118₂. The output load models liquids such as water vapor or particulates carrying pathogens as in 116. FIG. 3B illustrates an example implementation of the resonant circuit 300 of FIG. 3A.

In one embodiment, the input voltage Vin has a value of 50 V, the first capacitance C1 has a value of 5·10⁻¹² F, the second capacitance C2 has a value of 100·10⁻¹² F, the coupling factor K between primary and secondary has a value of 0.617, the primary inductance Lp has a value of 100·10⁻⁶ H, the secondary inductance Ls has a value of 4.3·10⁻³ H, the transformer primary resistance Rs has a value of 0.01Ω, the input impedance Rs2 has a value of 0.01Ω, the secondary resistance R3 has a value of 1Ω, and the output load R_load has a value of 10·10⁶Ω. It should be understood that the values of inductances, resistances and capacitances indicated above are for illustrative purposes only, for a particular application (e.g., a particular pathogen size range and air flow volume range). Other values of inductances, resistances and capacitances may be used to result in high voltages on the transformer's secondary winding. As used herein, the term “high voltage” refers to a voltage having a value that allows the desired high intensity alternating electric field within the device 100 to be achieved. Other embodiments of the resonant circuit 300 may therefore apply.

The resonant circuit 300 described above has an approximate power consumption of 75 W, with about 5.6 KVA being produced by the capacitive component 114 modeled by the second capacitor having capacitance C2. This indicates that large circulating energy fields may be produced within the device 100, without the direct dissipation of power, to thermally destroy any pathogen as in 116 passing through the capacitive component 114. The current and dielectric heating required to thermally destroy the pathogens 116 occur due to the intensity of the electric field generated by the capacitive component 114. Indeed, in a resonant system, the current is almost ninety degrees) (90° out of phase with the voltage, allowing to obtain large KVAs with little losses. The phase angle diverging from true 90° (π/2 Radians) corresponds to circuit losses, including power transmitted for dielectric heating to particulates in the air mass flowing through the concentric capacitive component 114. This phase angle loss component is commonly known as Tan(δ). As previously noted, large displacement currents within the capacitive component 114 (i.e. between the capacitor elements 118₁, 118₂) may be achieved using the device 100. In some embodiments, the device 100 allows for the creation of a destructive or damaging environment for airborne pathogens where a period of time to destroy pathogens (referred to as a “kill period”) in the order of milliseconds can be achieved, rather than minutes or hours as suggested by existing UVC and other devices.

In some embodiments, due to the high intensity alternating electric field generated by the capacitive component 114, the device 100 may be used to produce pathogen killing chemicals such as ozone (O₃) and hydrogen peroxide (H₂O₂). This adds to the device's efficacy of sterilization since ozone and hydrogen peroxide can be used to sterilize certain airborne and/or surface pathogens. Indeed, during operation of the device 100, ozone may be created in safe levels (e.g., below 0.05 ppm) and, due to the large current density of the electric field generated within the device 100 and to the potential existence of liquid droplets and/or solid particles in the surrounding air (e.g. humidity from water vapor/droplets carrying the pathogens 116 or introduced into the system via a separate feed such as an atomizer or source of liquid), unreacted oxygen radicals in the production of ozone may react with water to create hydrogen peroxide. The H₂O₂ production reaction is as follows:

At air-water interface

H₂O→H⁻|OH⁻OH^{\vec{E}.ΔpH}—OH⁻|e⁻(solv)OH⁻·OH⁻—H₂O₂ (1)

In one embodiment, the design of the device 100 may be optimized for production of either ozone and/or hydrogen peroxide and ozone and hydrogen peroxide levels may be increased for applications including, but not limited to, sterilization in hospitals. In such cases, sensors may be added to the device 100 to monitor the levels of ozone and hydrogen peroxide generated as well as detect occupation within a given area (e.g., hospital room) where the device 100 is provided. This may, for example, allow to prevent the device 100 from generating certain ozone and hydrogen peroxide levels, particularly when personnel are present. In some embodiments, in order to increase the efficacy of hydrogen peroxide production, alkaline metal catalysts including, but not limited to, carbon, may also be deposited on the surface of the capacitive component 114 exhibiting a high intensity alternating electric field (e.g. on the inner surface of the outer capacitor element 118₂ or on the outer surface of the separating member 120).

The systems and methods described herein may be used to sterilize pathogens including, but not limited to phi x 174 bacteriophage, Escherichia coli (E. coli), Staphylococcus epidermidis, SARS-COV-2, Bovine coronavirus (BCV or BCoV), Parvovirus, avian influenza virus, swine influenza virus (SIV) (or swine-origin influenza virus (S-OIV)), and other types of influenza viruses.

In order to evaluate the efficacy of the systems and methods described herein for sterilizing different pathogens, the sterilizing device (reference 100 in FIG. 1A) was placed in a chamber having a volume of 1000 cubic feet (e.g., having dimensions 10′×10′×10′). A microbial suspension was aspirated into the chamber. Air samples were taken from the chamber once the sterilizing device was turned on and sampling was taken every fifteen (15) minutes over a period of four (4) hours, and then plated. The process was then repeated without the sterilizing device in the chamber to provide natural decay results. All plates were incubated overnight and viral growth on the test plates was compared to that of the natural decay control.

In a first setting, the efficacy of the device for sterilizing phi×174 was tested. Table I below summarizes the results obtained (i.e. percent reduction at 240 minutes).

TABLE I

| | | | Natural | |
|---|---|---|---|---|
| Test Parameter | | Test Result | Decay Result | Units |
| Organism | Species | Phi-X174 | | — |
| | ATCC No. | 13706-B1 | | — |
| | Challenge | 1.0 × 10⁸ | | PFU/mL |
| | Concentration | | | |
| Samples | 0 | TNTC (2628) | TNTC (2628) | PFU |
| | 15 | TNTC (2628) | TNTC (2628) | PFU |
| | 30 | TNTC (2628) | TNTC (2628) | PFU |
| | 45 | TNTC (2628) | TNTC (2628) | PFU |
| | 60 | TNTC (2628) | TNTC (2628) | PFU |
| | 75 | TNTC (2628) | TNTC (2628) | PFU |
| | 90 | 149 | TNTC (2628) | PFU |
| | 105 | 103 | TNTC (2628) | PFU |
| | 120 | 38 | TNTC (2628) | PFU |
| | 135 | 5 | TNTC (2628) | PFU |
| | 150 | 1 | TNTC (2628) | PFU |
| | 165 | <1 | TNTC (2628) | PFU |
| | 180 | <1 | TNTC (2628) | PFU |
| | 195 | <1 | TNTC (2628) | PFU |
| | 210 | <1 | TNTC (2628) | PFU |
| | 225 | <1 | TNTC (2628) | PFU |
| | 240 | <1 | TNTC (2628) | PFU |
| Results | 240 Minutes | 99.9% | | Reduction |

* ATCC: American Type Culture Collection
* TNTC: Too Numerous To Count
* PFU: Plaque Forming Units

In a second setting, the efficacy of the device for sterilizing E. coli was tested. Table II below summarizes the results obtained (i.e. percent reduction at 120 minutes).

TABLE II

| | | | Natural | |
|---|---|---|---|---|
| | | | Decay | |
| Test Parameter | | Test Result | Result | Units |
| Organism | Species | Staphylococcus | | — |
| | | epidermidis | | |
| | ATCC No. | 12228 | | — |
| | Challenge | 3.4 × 10⁸ | | CFU/mL |
| | Concentration | | | |
| Samples | 0 | TNTC (2628) | TNTC (2628) | CFU |
| | 15 | TNTC (2628) | TNTC (2628) | CFU |
| | 30 | 189 | TNTC (2628) | CFU |
| | 45 | 47 | TNTC (2628) | CFU |
| | 60 | 14 | TNTC (2628) | CFU |
| | 75 | 2 | TNTC (2628) | CFU |
| | 90 | <1 | TNTC (2628) | CFU |
| | 105 | <1 | TNTC (2628) | CFU |
| | 120 | <1 | TNTC (2628) | CFU |
| Results | 75 Minutes | 99.9% | Reduction | Results |

* CFU: Colony Forming Units

In a first setting, the efficacy of the device for sterilizing Staphylococcus epidermidis was tested. Table III below summarizes the results obtained (i.e. percent reduction at 60 minutes).

TABLE III

| | | | Natural Decay | |
|---|---|---|---|---|
| | Test Parameter | Test Result | Result | Units |
| Organism | Species | E. Coli | | — |
| | ATCC No. | 11228 | | — |
| | Challenge | 2.3 × 10⁸ | | CFU/mL |
| | Concentration | | | |
| Samples | 0 | TNTC (2628) | TNTC (2628) | CFU |
| | 15 | 110 | TNTC (2628) | CFU |
| | 30 | 17 | TNTC (2628) | CFU |
| | 45 | 8 | TNTC (2628) | CFU |
| | 60 | <1 | TNTC (2628) | CFU |
| | 75 | <1 | 83 | CFU |
| | 90 | <1 | 49 | CFU |
| | 105 | <1 | 36 | CFU |
| | 120 | <1 | 27 | CFU |
| Results | 60 Minutes | 99.9% | Reduction | Results |

As can be seen from Tables I, II, and III, in one embodiment, a 99.9% reduction rate (i.e. kill efficacy) may be achieved on phi×174, E. coli, and Staphylococcus epidermidis.

In one embodiment, the use of the systems and methods described herein alleviates the need for filters, such as high-efficiency particulate air (HEPA) filters, that are typically used in UVC sterilization devices. Indeed, HEPA type filters typically have pores with a size of about 300 μm while some pathogens (e.g. viruses) can have a size significantly smaller than the pore size of a HEPA filter. As such, if a particulate or droplet carrying the pathogen is smaller than the filter's pore size, the particulate or droplet and virus is likely to pass through the filter. In those instances, existing UVC sterilization devices are caused to rely solely on the efficacy of UV radiation, thus reducing overall sterilizing efficacy.

Although some embodiments have been described in detail, it should be understood that various changes, substitutions and alterations can be made herein without departing from the scope as defined by the appended claims. In the context of the present disclosure, the expressions “about” and “substantially” include variations by plus or minus 10%.

Moreover, the scope of the present application is not intended to be limited to the particular embodiments of the process, machine, manufacture, composition of matter, means, methods and steps described in the specification. As one of ordinary skill in the art will readily appreciate from the present disclosure, other processes, machines, manufacture, compositions of matter, means, methods, or steps, that perform substantially the same function or achieve substantially the same result as the corresponding embodiments described herein, may be utilized. Accordingly, the appended claims are intended to include within their scope such processes, machines, manufacture, compositions of matter, means, methods, or steps.

What is claimed is:

1. A sterilization device for pathogens, comprising:
a housing having a first end and a second end opposite to the first end, the first end having formed therein at least one air inlet and the second end having formed therein at least one air outlet;
a capacitive component disposed within the housing and extending at least partially along an air flow path disposed between the at least one air inlet and the at least one air outlet;
a positive displacement device disposed within the housing and configured to direct a flow of air through the at least one air inlet and towards the capacitive component, the flow of air comprising one or more pathogens; and
a voltage source electrically connected to the capacitive component, the voltage source supplying an alternating voltage to the capacitive component for causing the capacitive component to generate an alternating electric field, the alternating electric field dielectrically heating the one or more pathogens for thermally destroying the one or more pathogens as the flow of air is directed towards the capacitive component.

2. The sterilization device of claim 1, where the voltage source is configured to supply the alternating voltage to the capacitive component for causing the capacitive component to generate the alternating electric field by electrical resonance.

3. The sterilization device of claim 1, wherein the one or more pathogens are destroyed by current induced through the one or more pathogens by the alternating electric field.

4. The sterilization device of claim 1, wherein a sterilized volume of air is produced at an exit of the capacitive component subsequent to the alternating electric field dielectrically heating the one or more pathogens for thermally destroying the one or more pathogens, and further wherein the air outlet is configured to discharge the sterilized volume of air therethrough.

5. The sterilization device of claim 1, wherein the capacitive component comprises a first capacitor element and at least one second capacitor element, the first capacitor element and the at least one second capacitor element being electrically conductive, the first capacitor element and the at least one second capacitor element arranged along the air flow path from the at least one air inlet to the at least one air outlet, the first capacitor element arranged within the at least one second capacitor element.

6. The sterilization device of claim 5, wherein the first capacitor element and the at least one second capacitor element are cylindrical-shaped and arranged concentrically.

7. The sterilization device of claim 5, wherein the first capacitor element and the at least one second capacitor element are plate-shaped.

8. The sterilization device of claim 6, further comprising a separating member interposed between the first capacitor element and the at least one second capacitor element for spacing the first capacitor element from the at least one second capacitor element.

9. The sterilization device of claim 8, wherein the separating member is an insulator.

10. The sterilization device of claim 6, wherein the separating member comprises a quartz tube.

11. The sterilization device of claim 5, wherein a blocking member is provided at an end of the first capacitor element, the end exposed to the flow of air directed by the positive displacement device towards the capacitive component.

12. The sterilization device of claim 11, wherein the blocking member is configured to prevent the flow of air from being directed into the first capacitor element via the end of the first capacitor element, and to allow the flow of air to be directed towards a space defined between an outer wall of the first capacitor element and an inner wall of the at least one second capacitor element.

13. The sterilization device of claim 12, wherein the alternating electric field is generated in the space defined between the outer wall of the first capacitor element and the inner wall of the at least one second capacitor element, and further wherein the blocking member is configured to prevent the flow of air from being directed into a space defined by inner walls of the first capacitor element and where no electric field is present to destroy the pathogens.

14. The sterilization device of claim 5, wherein the at least one second capacitor element comprises a single second capacitor element, and further wherein a first terminal of the voltage source is connected to the first capacitor element and a second terminal of the voltage source is connected to the second capacitor element.

15. The sterilization device of claim 5, wherein the at least one second capacitor element comprises a plurality of second capacitor elements, and further wherein terminals of the voltage source are connected to the first capacitor element and to the plurality of second capacitor elements in alternation.

16. The sterilization device of claim 1, wherein the alternating electric field generated by the capacitive component and liquid droplets and/or solid particles present in surrounding air cause at least one chemical compound to be produced within the housing, the at least one chemical compound adapted to destroy the one or more pathogens present in the flow of air and one or more pathogens present on at least one surface external to the device.

17. The sterilization device of claim 16, wherein the at least one chemical compound comprises ozone and/or hydrogen peroxide.

18. The sterilization device of claim 16, wherein the at least one chemical compound is adapted to be discharged through the at least one air outlet and to react with the one or more pathogens present on the at least one surface external to the device.

19. A method of sterilizing pathogens, comprising:
directing a flow of air through an air passage from an air inlet to an air outlet, the flow of air comprising one or more pathogens;
exposing the flow of air to an alternating electric field within the air passage between the air inlet and the air outlet, including generating the electric field by supplying an alternating voltage to a capacitive component, the capacitive component producing the alternating electric field; and
dielectrically heating the one or more pathogens present in the flow of air using the alternating electric field, to thermally destroy the one or more pathogens as the flow of air passes through the air passage.

20. A method comprising:
directing a flow of air through at least one air passage defined within a housing, the housing having a first end defining at least one air inlet and a second end opposite to the first end defining at least one air outlet; and
exposing the flow of air to an alternating electric field generated within the at least one air passage, between the at least one air inlet and the at least one air outlet, by supplying an alternating voltage to a capacitive component disposed within the housing, the capacitive component producing the alternating electric field, wherein, as the flow of air passes through the housing, one or more airborne pathogens present in the flow of air and/or one or more surface pathogens present on at least one surface of the housing are thermally destroyed by dielectric heating caused by the alternating electric field and chemically destroyed by at least one chemical compound produced by the alternating electric field and liquid droplets and/or solid particles present in surrounding air.
